(19) United States
(12) Patent Application Publication
(10) Pub. No.: US 2021/0156841 A1
(43) Pub. Date: May 27, 2021

(54) CYTODIAGNOSIS DEVICE AND CYTODIAGNOSIS METHOD

(71) Applicant: OLYMPUS CORPORATION, Tokyo (JP)

(72) Inventor: Takayasu MIKKAICHI, Tokyo (JP)

(73) Assignee: OLYMPUS CORPORATION, Tokyo (JP)

(21) Appl. No.: 17/163,665

(22) Filed: Feb. 1, 2021

Related U.S. Application Data
(63) Continuation of application No. PCT/JP2018/029227, filed on Aug. 3, 2018.

(51) Int. Cl. G01N 33/483; G01N 1/31; G01N 15/00

(52) U.S. Cl. CPC G01N 33/4833; G01N 2015/0065; G01N 15/00; G01N 1/31

(57) ABSTRACT

A cytodiagnosis method includes a preparation step, a first injection step, a first discharge step, a second injection step, a second discharge step, a contact step of bringing a filter and a pressing member into contact with each other after the second discharge step, a flattening step of flattening the fine undulations of the filter by pressing the filter with the pressing member after the contact step, and an observation step of observing the cells on the filter flattened by the flattening step with an observation unit while illuminating the filter with a light source while the filter is located between the light source and the observation unit.

### BACKGROUND OF THE INVENTION

### Field of the Invention

The present invention relates to a cytodiagnosis device and a cytodiagnosis method.

This application is a continuation application based on PCT/JP2018/029227, filed on Aug. 3, 2018. The content of the PCT Application is incorporated herein by reference.

### Description of Related Art

For the diagnosis of cancer and the like, a biopsy is widely performed in which a part of tissue is collected and a pathological diagnosis is performed. As a minimally invasive biopsy, a percutaneous puncturing suction needle biopsy or a biopsy performed with forceps and a puncturing suction needle using an endoscope or ultrasound endoscope (EBUS-GS, EBUS-TBNA, or EUS-FNA) is available. However, since an affected part is indirectly accessed by an ultrasound image, it is not always possible to reliably collect a target tissue. Thus, in some cases, rapid cell diagnosis (Rapid on-site evaluation, hereinafter referred to as ROSE) is performed to evaluate on the site whether the collected sample is a target sample. In general ROSE, a part of a sample collected at a bedside by a pathologist or cytotechnologist is isolated into a slide glass and is thinly spread. The part of sample is fixed on a slide by a rubbing method (smear method), cells are stained with a staining solution, and the cells are observed by a microscope to evaluate whether or not a target sample is collected. The user can be determined, at a location where the cells are collected, whether required cells are collected or not by the ROSE. In a case where the ROSE is not used, the cells collected by a puncture needle or the like are transported to a test location separate from the location where the cells have been collected, and tests such as pathological diagnosis are performed after a while.

As a result, the biopsy has already ended even in a case where the required cells have not been collected. In tests using the ROSE, it is possible to determine whether or not the required cells have been collected on the site where the cells have been collected. Therefore, the collection of the cells can be easily added. Therefore, since the ROSE can reduce the number of times of puncturing, reduce the burden on a patient, and reduce the re-test rate, it is desired to carry out the ROSE at each facility. However, since the ROSE is generally performed by a pathologist or a cytotechnologist, it is often extremely difficult to secure personnel. Additionally, respective steps are manually performed, and in particular, skill is required for producing smears and evaluating cell images. For that reason, facilities where implementation is possible are limited.

As a simpler method of cytology, there is liquid-based cytology (hereinafter referred to as LBC). A cell suspension is produced by storing the collected sample in a cell preservation solution and diffusing cells. After the cells are captured from the cell suspension and sampled, the cells are observed by a microscope. No special skills are required to produce the sample.

Although several kinds of methods for producing samples of cells by the LBC are present, as a method for producing the samples rapidly and easily, a method of capturing cells captured on a membrane filter and directly observing the cells with a microscope is known. However, in this case, when the cells are observed by the microscope, the pores (holes) of a filter are conspicuous and hinder the observation. For this reason, a prepared slide is disclosed in which a semi-transparent seal piece that scatters light is stuck on a transparent slide (see, for example, Japanese Unexamined Patent Application, First Publication No. H03-191310). In Japanese Unexamined Patent Application, First Publication No. H03-191310, the prepared slide is produced by forming an irregular part on the seal piece, fitting a frame, to which the filter is attached, to the irregular part, capturing cells captured on the filter, and placing a cover glass to which an enclosed agent is added, and the prepared slide is made.

Additionally, since the membrane filter is a thin film made of resin, the flatness thereof is not uniform, and the membrane filter swells due to an operation such as staining for producing a sample, and the flatness further deteriorates. For that reason, the operation of focusing the microscope becomes complicated.

### SUMMARY OF THE INVENTION

A cytodiagnosis method according to a first aspect of the present invention is a cytodiagnosis method for testing cells contained in a cell suspension by using a light source and an observation unit. The method includes a preparation step of disposing a chamber including a filter having a pore size and fine undulations between the light source and the observation unit, the pore size being capable of capturing the cells, the filter arranged in an internal space of the chamber; a first injection step of injecting the cell suspension into the internal space of the chamber from a front surface side of the filter; a first discharge step of discharging the cell suspension from a back surface side of the filter to an outside of the chamber; a second injection step of injecting a staining solution for staining the cells into the internal space of the chamber from the front surface side of the filter; a second discharge step of discharging the staining solution from the back surface side of the filter to the outside of the chamber; a flattening step of flattening the filter by pressing the filter with the pressing member located between the light source and the observation unit; and an observation step of observing the cells on the filter flattened by the flattening step with the observation unit while illuminating the filter with the light source while the filter is located between the light source and the observation unit.

In the cytodiagnosis method according to a second aspect of the present invention on the basis of the first aspect, the cell suspension and the staining solution may be injected into the internal space from a side wall of the chamber.

In the cytodiagnosis method according to a third aspect of the present invention on the basis of the first aspect or the second aspect, the cell suspension and the staining solution may be discharged from a side wall of the chamber to the outside of the internal space.

The cytodiagnosis method according to a fourth aspect of the present invention on the basis of any one of the first aspect to the third aspect, in the first injection step and the second injection step, a space which is disposed on the front surface side of the filter may be formed, the space may be surrounded by the pressing member, an inner wall surface of the chamber and the filter, and the space in which the cell suspension and the staining solution are injected may be formed in the front surface side of the filter.

The cytodiagnosis method according to a fifth aspect of the present invention on the basis of any one of the first aspect to the third aspect, in the first discharge step and the second discharge step, a space which is disposed on the back surface side of the filter may be formed, the space may be surrounded by the pressing member, an inner wall surface of the chamber and the filter, and the space in which the cell suspension and the staining solution are discharged may be formed in the back surface side of the filter.

In the cytodiagnosis method according to a sixth aspect of the present invention on the basis of the fourth aspect or the fifth aspect, in the observation step, a space between the filter and the pressing member may be filled with a liquid.

In the cytodiagnosis method according to a seventh aspect of the present invention on the basis of any one of the fourth aspect to the sixth aspect, in the observation step, the cells on the filter may be observed by the observation unit through a cover glass provided between the observation unit and the filter.

In the cytodiagnosis method according to an eighth aspect of the present invention on the basis of any one of the fourth aspect to the seventh aspect, the pressing member may be made of a light-transmitting material through which light from the light source is transmitted.

A cytodiagnosis device according to a ninth aspect of the present invention a filter having a pore size that is capable of capturing cells contained in a cell suspension and fine undulations; a chamber having an internal space and having the filter disposed in the internal space; an injection unit that injects the cell suspension and a staining solution for staining the cells into the internal space of the chamber from a front surface side of the filter; a discharge unit that is configured to discharge the cell suspension and the staining solution from a back surface side of the filter to an outside of the chamber; a pressing member that is provided in the internal space so as to be movable relative to the filter; a control unit that is configured to control a movement of the pressing member;

a light source that is configured to irradiate the cells with light; and an observation unit that is provided on a side opposite to the light source with respect to the filter. The control unit is configured to bring the pressing member into contact with the filter by moving the pressing member to the filter in a direction in which the pressing member and the filter approach each other, and to press the filter by the pressing member, and the undulations of the filter are flattened.

In the cytodiagnosis device according to a tenth aspect of the present invention on the basis of the ninth aspect, the light source may be provided so as to be movable relative to the observation unit, and the light source may be configured to push any one of the pressing member and the filter in a direction approaching the other.

In the cytodiagnosis device according to an eleventh aspect of the present invention on the basis of the ninth aspect, the pressing member may be provided in the chamber and is disposed on the front surface side or the back surface side of the filter, and a space which is surrounded by the pressing member, an inner wall surface of the chamber and the filter may be formed, the cell suspension and the staining solution injected into the space.

In the cytodiagnosis device according to a twelfth aspect of the present invention on the basis of in any one of the ninth aspect to the eleventh aspect, the injection unit and the discharge unit may be attachable to and detachable from the chamber.

In the cytodiagnosis device according to a thirteenth aspect of the present invention on the basis of any one of the ninth aspect to the twelfth aspect, the pressing member may be made of a light-transmitting material through which light from the light source is transmitted.

In the cytodiagnosis device according to a fourteenth aspect of the present invention on the basis of any one of the ninth aspect to the thirteenth aspect, the pressing member may be a diffusion plate that is configured to diffuse light from the light source.

In the cytodiagnosis device according to a fifteenth aspect of the present invention on the basis of any one of the ninth aspect to the fourteenth aspect, a cover glass may be provided between the observation unit and the filter.

### BRIEF DESCRIPTION OF THE DRAWINGS

FIG. 1 is an overall view illustrating a cytodiagnosis device according to a first embodiment of the present invention.

FIG. 2 is a perspective view illustrating a filter of FIG. 1.

FIG. 3 is a sectional view of main parts illustrating a state when the filter of FIG. 1 is pressed by a pressing member.

FIG. 4 is a block diagram illustrating the cytodiagnosis device of the present invention.

FIG. 5 is a flowchart illustrating a cytodiagnosis method of the present invention.

FIG. 6 is a view illustrating a procedure for producing a cell suspension used in the cytodiagnosis method of the present invention.

FIG. 7 is a view illustrating the procedure for producing the cell suspension used in the cytodiagnosis method of the present invention.

FIG. 8 is a view illustrating the procedure for producing the cell suspension used in the cytodiagnosis method of the present invention.

FIG. 9 is a view illustrating the procedure for producing the cell suspension used in the cytodiagnosis method of the present invention.

FIG. 10 is a view illustrating the cytodiagnosis method of the present invention.

FIG. 11 is a view illustrating the cytodiagnosis method of the present invention.

FIG. 12 is a view illustrating the cytodiagnosis method of the present invention.

FIG. 13 is a view illustrating the cytodiagnosis method of the present invention.

FIG. 14 is a view illustrating a cytodiagnosis method of the present invention.

FIG. 15 is a view illustrating a modification example of the cytodiagnosis device of the first embodiment of the present invention.

FIG. 16 is an overall view illustrating a cytodiagnosis device according to a second embodiment of the present invention.

FIG. 17 is a view illustrating the cytodiagnosis method of the present invention.

FIG. 18 is a view illustrating the cytodiagnosis method of the present invention.

### DETAILED DESCRIPTION OF THE INVENTION

### First Embodiment

A cytodiagnosis device according to a first embodiment of the present invention will be described with reference to FIGS. 1 to 14.

The cytodiagnosis device 1 is a device that captures and observes cells contained in a cell suspension. As illustrated in FIG. 1, the cytodiagnosis device 1 includes a filter 10, a chamber 20, an injection unit 30, a discharge unit 40, a pressing member 50, a light source 60, and an observation unit 70. The filter 10, the chamber 20, and the pressing member 50 are disposed between the light source 60 and the observation unit 70.

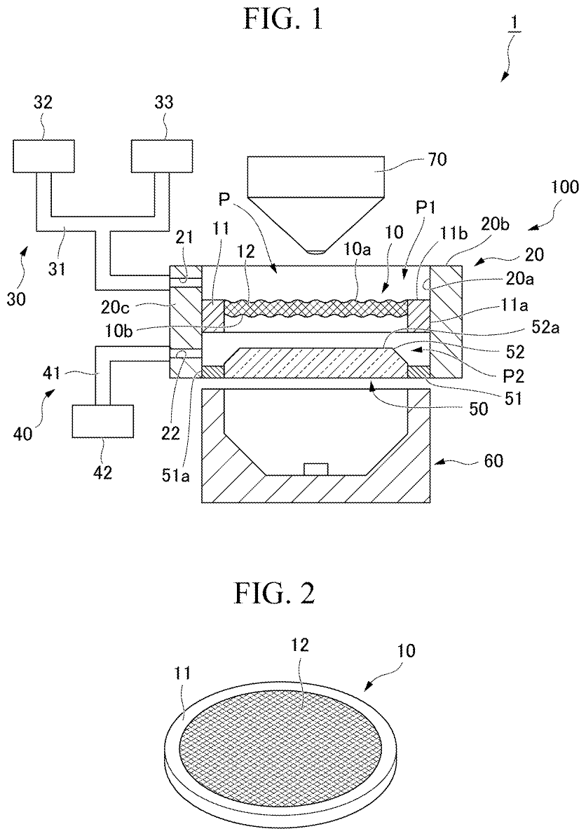

As illustrated in FIG. 2, the filter 10 includes an annular frame 11 and a filter part 12 that captures cells stuck to a front surface of the frame 11. The filter part 12 has a pore size smaller than that of the cells in order to capture the cells contained in the cell suspension. A filter medium of the filter part 12 is a thin film membrane filter, and a filter having independent holes of a resin such as polycarbonate is often used. Since the filter part 12 is a thin film, the filter part 12 usually has fine undulations rather than a flat surface. In FIG. 1, the undulations are illustrated in an enlarged manner for easy understanding.

As illustrated in FIG. 1, the chamber 20 is disposed between the light source 60 and the observation unit 70. The chamber 20 has an internal space P, and the filter 10 is disposed in the internal space P. An outer surface 11a of the frame 11 of the filter 10 is in contact with an inner wall surface 20a of the chamber 20 in an airtight and watertight manner. The filter 10 is movably disposed in the internal space P while the frame 11 of the filter 10 is in contact with the inner wall surface 20a of the chamber 20. The filter 10 is movable until a front surface 11b of the frame 11 of the filter 10 and a front surface 20b of the chamber are flush with each other. A stopper is provided on the front surface 20b of the chamber to restrict the movement of the filter 10 in a direction toward the observation unit 70.

A first through-hole 21 and a second through-hole 22 are formed at a distance in an arrangement direction of the observation unit 70 and the light source 60 on a side wall 20c of the chamber 20. The first through-hole 21 is disposed closer to the observation unit 70 than the second through-hole 22. The second through-hole 22 is disposed closer to the light source 60 than the first through-hole 21. The first through-hole 21 and the second through-hole 22 allow the internal space P of the chamber 20 to communicate with the outside.

The filter 10 is disposed at a position between the first through-hole 21 and the second through-hole 22 in the internal space P when the chamber 20 is viewed from a side. An upper space P1, which is surrounded by the filter 10 and the inner wall surface 20a and the front surface 20b of the chamber 20, is formed in the internal space P.

As illustrated in FIG. 1, the injection unit 30 includes a flow path 31 communicating with the first through-hole 21, an injection port 32 through which the cell suspension C is injected into the internal space P, and a storage part 33 in which a staining solution S is stored. The storage part includes a plurality of the staining solutions and a cleaning solution. The injection unit 30 injects a cell suspension C and a staining solution S, which stains the cells from the front surface 10a side of the filter 10, into the upper space P1 of the chamber 20 via the flow path 31. In FIG. 1, although the flow path 31 is integrated into one and the first through-hole 21 is one, the flow path 31 may be independent for each of the cell suspension C, the plurality of staining solutions S, and the cleaning solution, and correspondingly, a plurality of the first through-holes 21 may be provided. Each solution in the storage part 33 is independently injected into the upper space P1 of the chamber 20 by a piezo actuator, a stepping motor, or the like of a main body.

As illustrated in FIG. 1, the discharge unit 40 includes a flow path 41 attached to the second through-hole 22 and a suction part 42 for suctioning the cell suspension C and the staining solution S. The discharge unit 40 discharges the cell suspension C and the staining solution S in the internal space P from a back surface 10b side of the filter 10 to the outside of the chamber 20 via the flow path 41 by the piezo actuator or the stepping motor of the main body.

As illustrated in FIG. 1, the pressing member 50 is disposed on the back surface 10b side of the filter 10 in the internal space P and is provided so as to be movable relative to the filter 10. The pressing member 50 is disposed closer to the light source 60 side than the second through-hole 22 at a distance from the filter 10 in the internal space P. A lower space (space) P2, which is surrounded by the pressing member 50, the inner wall surface 20a of the chamber 20, and the filter 10 and is adapted to discharge the cell suspension C and the staining solution S to the back surface 10b side of the filter 10 therefrom outside the chamber 20, is formed in the internal space P.

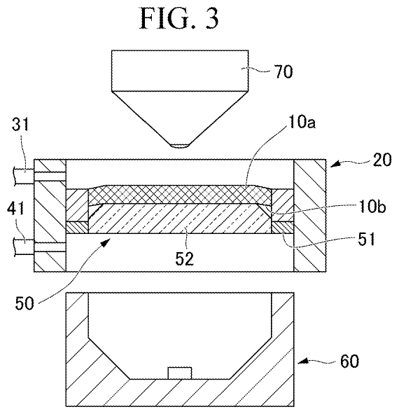

The pressing member 50 includes an annular frame 51 and a protruding member 52 that is disposed in the frame 51 and comes into contact with the filter 10. A surface 52a of the protruding member 52 facing the filter 10 is a smooth flat surface. The diameter of the surface 52a is smaller than the diameter of the filter. An outer surface 51a of the frame 51 of the pressing member 50 is in contact with the inner wall surface 20a of the chamber 20 in an airtight and watertight manner. The pressing member 50 is movable in the internal space P while the frame 51 of the pressing member 50 is in contact with the inner wall surface 20a of the chamber 20. As illustrated in FIG. 3, the protruding member 52 of the pressing member 50 presses the back surface 10b of the filter 10, so that tension is applied to the filter 10 and the front surface 10a becomes flat.

The protruding member 52 is made of a light-transmitting material that transmits light from the light source 60. Additionally, the protruding member 52 scatters the light from the light source 60. The protruding member 52 may be formed of, for example, a plurality of transparent members having different refractive indexes from each other or may be formed of a scattering plate such as ground glass or a diffusion plate having minute irregularities disposed on the front surface.

The light source 60 is connected to, for example, the piezo actuator or the stepping motor and is movable in a direction closer to the observation unit 70 or in a direction away from the observation unit 70. The size of the light source 60 is substantially the same as the size of the frame 51 of the pressing member 50. The light source 60 is movable in the internal space P, and the pressing member 50 can be pushed toward the back surface 10b of the filter 10.

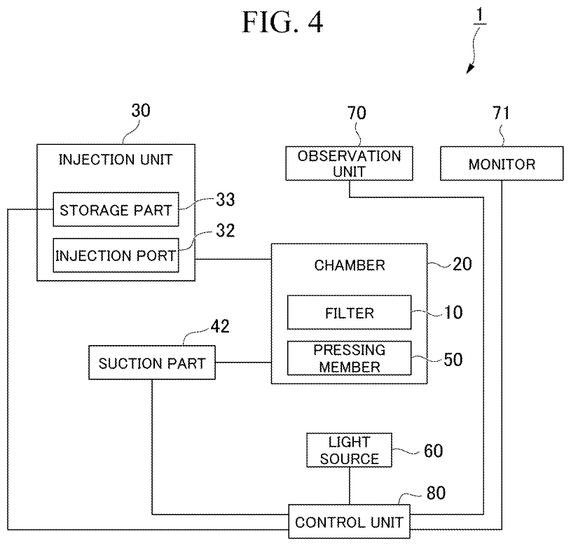

Next, a block configuration of the cytodiagnosis device 1 will be described. FIG. 4 is a block diagram of the cytodiagnosis device 1.

As illustrated in FIG. 4, the cytodiagnosis device 1 includes a control unit 80. The control unit 80 has a microscope image processing and storing mechanism built therein and is connected to an injection mechanism of the storage part 33, a suction mechanism of the suction part 42, a light source 60, and a monitor 71. The control unit 80 controls the operations of the injection mechanism of the storage part 33, the suction mechanism of the suction part 42, the observation unit 70, and the light source 60 depending on a command signal input by an input unit (not illustrated).

When a command signal for injecting the staining solution S is input, the control unit 80 controls the driving of the injection mechanism of the storage part 33, and the type and amount of staining solution S and cleaning solution according to the input command signal are injected into the internal space P of the chamber 20 via the flow path 31 with the times according to the command signal.

When a command signal for suctioning the cell suspension C or the staining solution S and the cleaning solution is input, the control unit 80 controls the driving of the suction mechanism of the suction part 42, and the cell suspension C or the staining solution S and the cleaning solution in the internal space P of the chamber 20 are suctioned by the suction part 42.

When a command signal for moving the light source 60 is input, the control unit 80 controls the driving of the light source 60 and moves the light source 60 in the direction closer to the observation unit 70.

The cytodiagnosis device 1 further includes a monitor (display unit) 71. The monitor 71 is connected to the control unit 80. An image of cells Tb acquired by the observation unit 70 is appropriately image-processed by the control unit 80 and then displayed on the monitor 71.

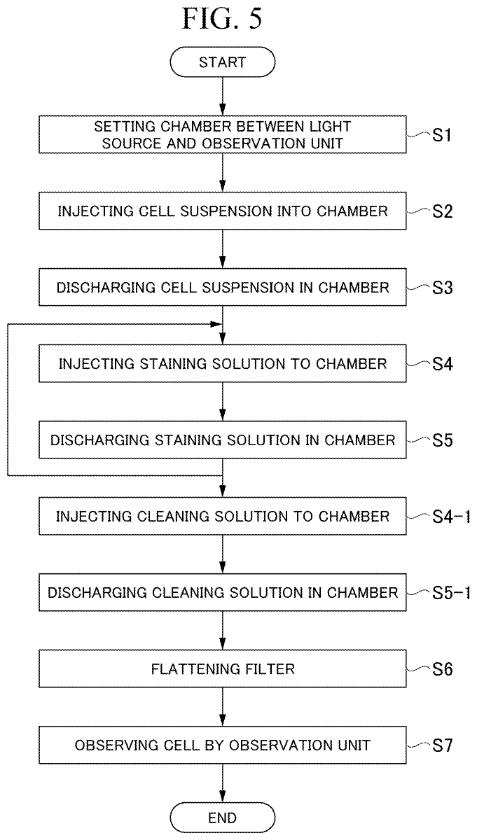

Next, the cytodiagnosis method according to the present embodiment will be described with reference to a flowchart of FIG. 5.

First, as illustrated in FIG. 1, the chamber 20 including the filter 10 and the pressing member 50 in the internal space, and a cartridge having the injection unit 30 and the discharge unit 40 are disposed between the light source 60 and the observation unit 70 (preparation step: Step S1).

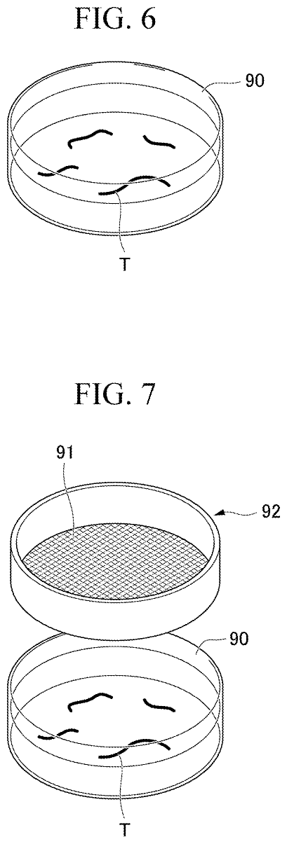

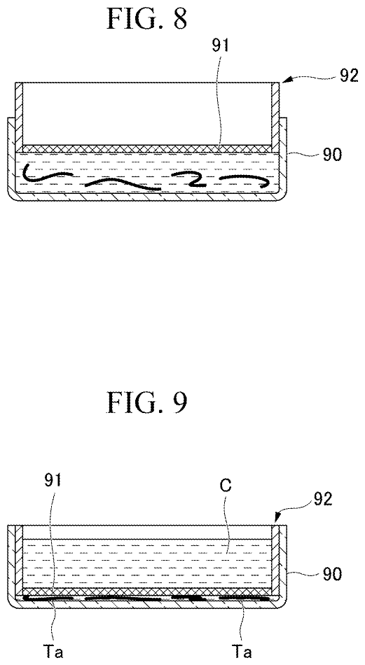

Next, while observing the inside of a patient's body with an ultrasound endoscope, a puncture needle or the like is inserted into a tissue to collect cells. As illustrated in FIG. 6, a collected sample T is discharged into a petri dish 90, and a cell preservation solution is injected into the petri dish 90 to loosen the sample T. After the sample T is loosened, as illustrated in FIG. 7, a container 92 having a filter 91 provided at a bottom part thereof is inserted into the petri dish 90. As illustrated in FIG. 8, when the container 92 is pushed into the petri dish 90, the cell suspension C in the container 92 and a tissue Ta in the petri dish 90 are separated from each other as illustrated in FIG. 9. In this way, the cell suspension C is produced.

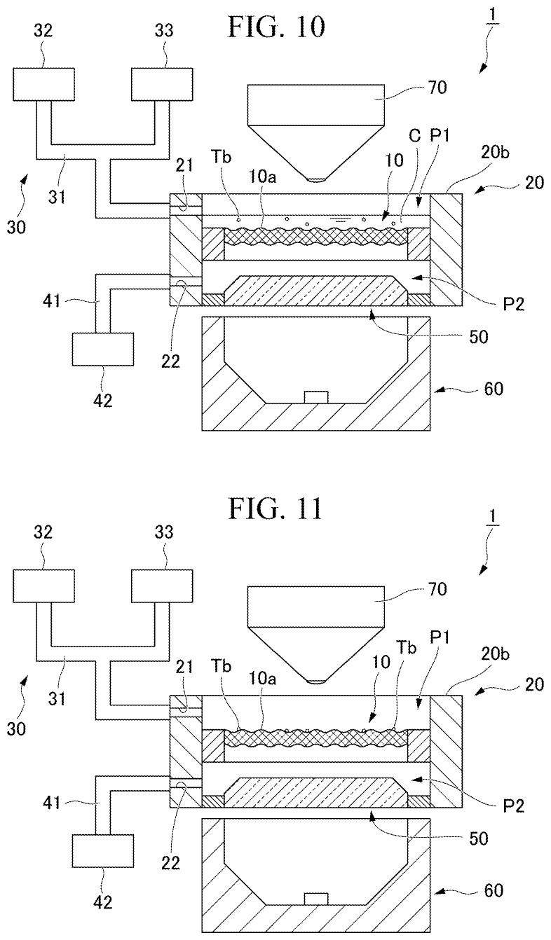

The produced cell suspension C is suctioned into a dropper, a syringe, or the like and injected into the injection port 32 illustrated in FIG. 1. The cell suspension C is injected from the first through-hole 21 through the flow path 31 into the upper space P1 of the chamber 20 (first injection step: Step S2). As illustrated in FIG. 10, the cell suspension C stays on the front surface 10a of the filter 10.

Next, the control unit 80 drives the suction mechanism of the suction part 42 to create a negative pressure in the lower space P2, thereby suctioning the cell suspension C in the upper space P1 and the cell suspension C that has passed through the filter 10. The suctioned cell suspension C is discharged from the second through-hole 22 through the flow path 41 to the suction part 42 (first discharge step: Step S3). As illustrated in FIG. 11, the cells in the cell suspension C are captured on the filter 10.

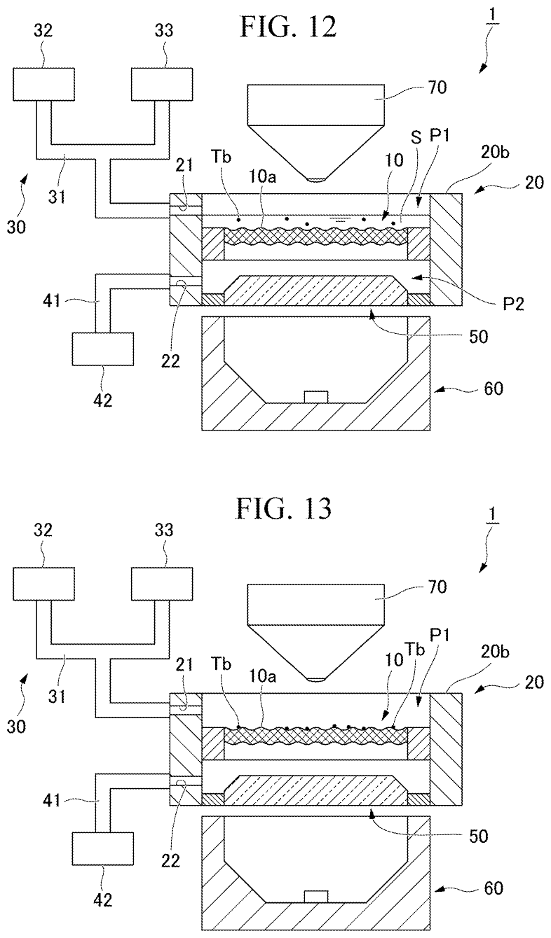

Next, the control unit 80 drives the injection mechanism of the storage part 33. The staining solution S and the cleaning solution in the storage part 33 are injected from the first through-hole 21 through the flow path 31 into the upper space P1 of the chamber 20 (second injection step: Step S4). As illustrated in FIG. 12, the staining solution C stays on the front surface 10a of the filter 10 where the cells Tb are stained. In addition, in the first injection step and the second injection step, in a case where the filter 10 is located closer to the observation unit 70 than the first through-hole 21 or in a case where the first through-hole 21 is blocked, the filter 10 is disposed closer to the light source 60 side than the first through-hole 21 to form the internal space P1 into which the cell suspension C and the staining solution S are injected.

Next, the control unit 80 drives the suction mechanism of the suction part 42 to create a negative pressure in the lower space P2, thereby suctioning the staining solution S in the upper space P1 and the staining solution S that has passed through the filter 10. The suctioned staining solution S is discharged from the second through-hole 22 through the flow path 41 to the suction part 42 (second discharge step: Step S5). A staining solution S different from the earlier is injected from the flow path 31 (Step S4) and discharged from the flow path 41 (Step S5). The injection (Step S4) and the discharge (Step S5) of the staining solution S are performed by the number of types of staining solutions.

As illustrated in FIG. 13, the cells Tb are stained. In addition, in the first discharge step and the second discharge step, in a case where the filter 10 is located closer to the light source 60 than the second through-hole 22 or in a case where the second through-hole 22 is blocked, the filter 10 is disposed closer to the observation unit 70 side than the second through-hole 22, and an internal space P2 into which the cell suspension C and the staining solution S are discharged is formed. After all the staining is completed, the cleaning solution is injected into the chamber 20 in the same step (Step S4-1), and then the cleaning solution is discharged (Step S5-1) to clean the excess staining solution.

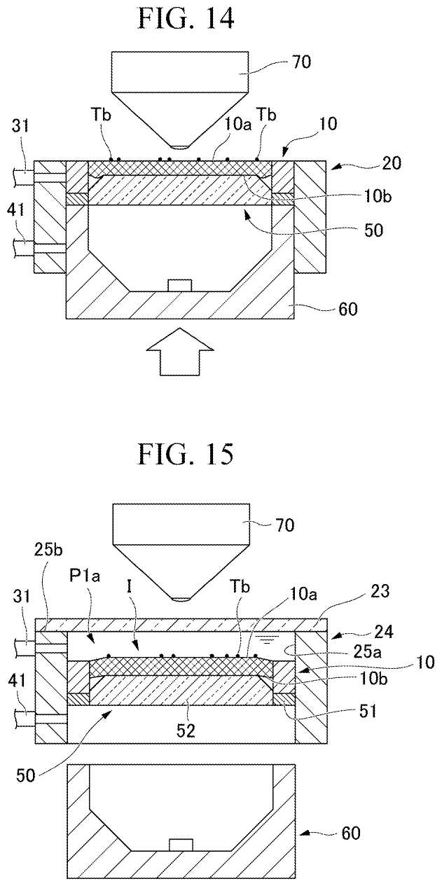

Next, when the control unit 80 drives the light source 60, the light source 60 moves in a direction closer to the pressing member 50. The light source 60 comes into contact with the pressing member 50 and moves toward the filter 10 together with the pressing member 50. Moreover, when the light source 60 moves in a direction closer to the filter 10, the protruding member 52 of the pressing member 50 comes into contact with the back surface 10b of the filter 10. The light source 60 pushes the pressing member 50 and the filter 10 until the front surface 10a of the filter 10 and the front surface 20b of the chamber are flush with each other. When the pressing member 50 is pressed against the back surface 10b of the filter 10, as illustrated in FIG. 14, the protruding member 52 of the pressing member 50 presses the filter 10. Therefore, tension is applied to the filter 10 that has captured the cells Tb, and the front surface becomes flat (flattening step: Step S6).

While the flattened filter 10 is illuminated with light from the light source 60, the observation unit 70 observes the cells on the front surface 10a of the filter 10 on the monitor 71 (observation step: Step S7). In a case where required cells have been collected, the tissue Ta and the remaining cell suspension C, which are illustrated in FIG. 9, are transported for the pathological examination. On the other hand, in a case where the required cells have not been collected, a sample is collected again using the endoscope.

According to the cytodiagnosis device 1 of the present embodiment, the filter 10 can be flattened by using the pressing member 50. Accordingly, it is possible to accurately observe the cells Tb captured on the front surface 10a of the filter 10. That is, usually, in a case where the cells on the filter 10 are observed, the filter 10 undulates. Therefore, the observation unit 70 is focused only on some of the cells Tb, and it is difficult to observe the entire cells Tb. In the present embodiment, since the filter 10 can be flattened, it is easy to focus on the cells Tb, and a clear image of the cells Tb can be observed.

Additionally, the lower space P2, which is surrounded by the pressing member 50, the inner wall surface 20a of the chamber 20, and the filter 10 and is adapted to discharge the cell suspension C and the staining solution S to the back surface 10b side of the filter 10, is formed in the internal space P of the chamber 20. Accordingly, the filter 10 and the pressing member 50 do not hinder the suction of the cell suspension C and the staining solution S.

Additionally, the protruding member 52 of the pressing member 50 is made of a transmitting member that transmits light from the light source 60. Accordingly, since the light from the light source 60 can be efficiently used, the cells Tb can be clearly observed by the observation unit 70. Additionally, the protruding member 52 of the pressing member 50 is a scattering plate that scatters the light from the light source 60. Accordingly, since it is not necessary to provide the scattering plate in addition to the pressing member 50, the number of parts can be reduced.

Additionally, the cartridge 100 constituted by the chamber 20, the injection unit 30, and the discharge unit 40 can be configured as a disposable type that is replaced for each use. Accordingly, the device is not contaminated by a sample, and infection caused by a sample of a medical worker and contamination between samples can also be prevented.

Additionally, according to the cytodiagnosis method of the present embodiment, the preparation step (Step S1), the first injection step (Step S2), the first discharge step (Step S3), the second injection step (Step S4), the second discharge step (Step S5), the flattening step (Step S6), and the observation step (Step S7) are performed between the light source 60 and the observation unit 70, that is, at the same location. Therefore, the cells Tb can be smoothly and rapidly tested.

Additionally, by forming the first through-hole 21 in the side wall 20c of the chamber 20, the cell suspension C and the staining solution S can be injected into the upper space P1 without disturbing the observation unit 70. By forming the second through-hole 22 in the side wall 20c of the chamber 20, the cell suspension C and the staining solution S can be discharged to the outside from the lower space P2 without disturbing the light source 60.

In addition, the pressing member 50 may not necessarily be made of a light-transmitting material but is preferably made of a member having a high light transmittance. The pressing member 50 may not necessarily be a diffusion plate.

Additionally, although a plurality of types of staining solutions S are used, only one type may be used. Cleaning using the cleaning solution is not always necessary.

Additionally, although the light source 60 pushes the pressing member 50, a configuration may be adopted in which the light source 60 does not move and the pressing member 50 is connected to an actuator or the like to move.

Additionally, although the control unit 80 drives the injection mechanism of the storage part 33, the suction mechanism of the suction part 42, and the light source 60, the control unit 80 may not be provided and the storage part 33, the suction part 42, and the light source 60 are manually operated. Although the control unit 80 controls the injection mechanism of the storage part 33, the suction mechanism of the suction part 42, and the light source 60 by inputting a command signal, all a series of flows may proceed automatically.

Additionally, although the image of the cells Tb acquired by the observation unit 70 is observed on the monitor 71, the cells Tb may be visually observed through an eyepiece.

Additionally, although the front surface 20b of the chamber is provided with the stopper for restricting the movement of the filter 10 in the direction toward the observation unit 70, a configuration may be adopted in which the light source 60 is moved by a movement amount preset by the control unit 80.

### Modification Example

In the present modification example, the configuration of a chamber 24 is different from the configuration of the chamber 20 of the first embodiment. The same constituent elements as those described above will be designated by the same reference signs, and a duplicate description will be omitted.

In the chamber 24, as illustrated in FIG. 15, a cover glass 23 is provided on a front surface 25b of the chamber 24. An upper space P1a surrounded by the filter 10, an inner wall surface 25a of the chamber 24, and the cover glass 23 is formed. The upper space P1a is hermetically sealed.

Next, the cytodiagnosis method will be described.

First, the above-described Steps S1 to S6 are performed. An enclosed liquid I is injected into the upper space P1a via the flow path 31. The cells on the front surface 10a of the filter 10 are observed on the monitor 71 by the observation unit 70 while the flattened filter 10 is illuminated with light by the light source 60 in a state where the upper space P1a is filled.

In addition, the cells Tb are observed at a distance between the filter 10 and the cover glass 23. However, even when the filter 10 is brought close to the cover glass 23 to narrow the space, the cells may be observed in contact.

Additionally, a transparent resin plate may be used instead of the cover glass 23. By filling the upper space P1a with the enclosed liquid, it is possible to perform the observation with the influence of the pores of the filter 10 on an image further reduced.

### Second Embodiment

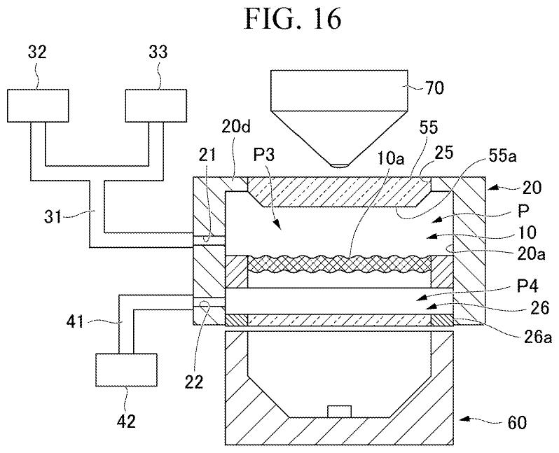

A second embodiment of the present invention will be described with reference to FIGS. 16 to 18.

The cytodiagnosis device of the present embodiment is different from the first embodiment in the configuration of the pressing member.

In the following description, the same constituent elements as those described above will be designated by the same reference signs, and a duplicate description will be omitted.

As illustrated in FIG. 16, an opening part 25 is formed in an upper wall 20d of the chamber 20. The pressing member 55 is provided at the position of the upper wall 20d of the chamber 20 facing the observation unit 70 so as to block the opening part 25. A surface 55a (a surface on the internal space P side) of the pressing member 55 facing the filter 10 is a flat surface. The pressing member 55 is made of a light-transmitting material that transmits light from the light source 60.

Similar to the first embodiment, the filter 10 is disposed at a position between the first through-hole 21 and the second through-hole 22 w in the internal space P hen the chamber 20 is viewed from the side. An upper space (space) P3 surrounded by the filter 10, the inner wall surface 20a of the chamber 20, and the pressing member 55 is formed in the internal space P.

In the internal space P of the chamber 20, a diffusion plate 26 is provided on the back surface 10b side of the filter 10. The diffusion plate 26 is disposed closer to the light source 60 than the second through-hole 22. A lower space P4 surrounded by the diffusion plate 26, the inner wall surface 20a of the chamber 20, and the filter 10 is formed in the internal space P. An outer surface 26a of the diffusion plate 26 is disposed in contact with the inner wall surface 20a of the chamber 20. The diffusion plate 26 is movable in the internal space P while the outer surface 26a is in contact with the inner wall surface 20a of the chamber 20. The diffusion plate 26 diffuses the light from the light source 60.

Similar to the first embodiment, the light source 60 is movable in the internal space P and is capable of pushing the diffusion plate 26 toward the back surface 10b of the filter 10.

The storage part 33 includes the enclosed liquid I in addition to the staining solution S and the cleaning solution.

Next, the cytodiagnosis method according to the present embodiment will be described with reference to a flowchart illustrated in FIG. 5.

The description of the same parts as those in the first embodiment will be omitted.

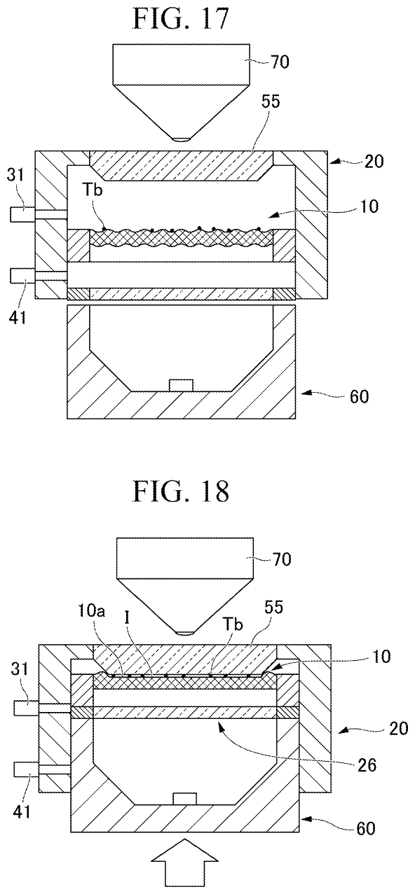

The pressing member 55 is provided on the upper wall 20d between the light source 60 and the observation unit 70, and the chamber 20 including the filter 10 and the diffusion plate 26 is disposed in the internal space P (preparation step: Step S1). The cell suspension C is injected into the upper space P3 (first injection step: Step S2), the cell suspension C is discharged with negative pressure in the lower space P4 (first discharge step: Step S3), and the cells Tb are captured on the filter 10. Thereafter, the staining solution S is injected into the upper space P3 (second injection step: Step S4), the staining solution S is discharged with negative pressure in the lower space P4 (second discharge step: Step S5), and the cells Tb are stained. As illustrated in FIG. 17, the stained cells Tb are captured on the front surface 10a of the filter 10. Next, a small amount of the enclosed liquid (liquid) I is injected onto a filter, on which the cells Tb stained with the staining solution are placed, to moisten the filter.

Next, when the control unit 80 drives the light source 60, the light source 60 moves in a direction closer to the diffusion plate 26. The light source 60 comes into contact with the diffusion plate 26 and moves toward the filter 10 together with the diffusion plate 26. Moreover, when the light source 60 moves in the direction closer to the filter 10, the filter 10 and the diffusion plate 26 come into contact with each other. The light source 60 pushes the filter 10 and the diffusion plate 26 toward the pressing member 55. When the filter 10 is pressed by the pressing member 55, as illustrated in FIG. 18, the filter 10 that has captured the cells Tb is flattened (flattening step: Step S6). The space between the pressing member 55 and the front surface 10a of the filter 10 is filled with the enclosed liquid I. In FIG. 18, the cells Tb captured on the filter 10 are displayed in an enlarged manner for easy understanding.

Next, similar to the first embodiment, the cells Tb are observed by the observation unit 70 (observation step: Step S7). In a case where the required cells have been collected, cells Ta illustrated in FIG. 9 are transported for the pathological examination. On the other hand, in a case where the required cells have not been collected, the cells are collected again using the endoscope.

According to the cytodiagnosis device of the present embodiment, the filter 10 can be flattened by the pressing member 55. Accordingly, it is possible to accurately observe the cells Tb captured on the front surface 10a of the filter 10.

Additionally, the upper space P3, which is surrounded by the pressing member 55, the inner wall surface 20a of the chamber 20, and the filter 10 and is adapted to inject the cell suspension C and the staining solution S to the front surface 10a side of the filter 10, is formed in the internal space P of the chamber 20. Accordingly, the filter 10 and the pressing member 55 do not hinder the injection of the cell suspension C and the staining solution S.

Additionally, in the observation step, since the space between the filter 10 and the pressing member 55 is filled with the enclosed liquid I and the enclosed liquid enters the pores of the filter, the influence of the pores can be reduced and the cells Tb can be clearly observed. Additionally, in a case where the space between the filter 10 and the pressing member 55 is not filled with a liquid, there is a case where sticking or floating of bubbles or the filter occurs between the filter 10 and the pressing member 55, making it difficult to observe.

In addition, the space between the filter 10 and the pressing member is filled with the enclosed liquid I. Usually, a liquid such as xylene adjusted to have such a refractive index that the influence of the pores does not easily appear during microscope observation is used as the enclosed liquid. However, the type of enclosed liquid such as alcohol, glycerin solution, and water is not particularly limited. For example, when the cleaning solution after the staining solution is suctioned, the cleaning solution may not be all discharged but may be slightly left.

Additionally, the diffusion plate 26 may not be necessarily provided, and the light source 60 may directly press the filter 10 and press the filter 10 against the pressing member 55.

While preferred embodiments of the invention have been described and illustrated above, it should be understood that these are exemplary of the invention and are not to be considered as limiting. Additions, omissions, substitutions, and other modifications can be made without departing from the scope of the invention. Accordingly, the invention is not to be considered as being limited by the foregoing description, and is only limited by the scope of the appended claims.

What is claimed is:

1. A cytodiagnosis method for testing cells contained in a cell suspension by using a light source and an observation unit, the method comprising:
a preparation step of disposing a chamber including a filter having a pore size and fine undulations between the light source and the observation unit, the pore size being capable of capturing the cells, the filter arranged in an internal space of the chamber;
a first injection step of injecting the cell suspension into the internal space of the chamber from a front surface side of the filter;
a first discharge step of discharging the cell suspension from a back surface side of the filter to an outside of the chamber;
a second injection step of injecting a staining solution for staining the cells into the internal space of the chamber from the front surface side of the filter;
a second discharge step of discharging the staining solution from the back surface side of the filter to the outside of the chamber;
a contact step of bringing the filter and a pressing member into contact with each other after the second discharge step;
a flattening step of flattening the fine undulations of the filter by pressing the filter with the pressing member after the contact step; and
an observation step of observing the cells on the filter flattened by the flattening step with the observation unit while illuminating the filter with the light source while the filter is located between the light source and the observation unit.

2. The cytodiagnosis method according to claim 1,
wherein the cell suspension and the staining solution are injected into the internal space from a side wall of the chamber.

3. The cytodiagnosis method according to claim 1,
wherein the cell suspension and the staining solution are discharged from a side wall of the chamber to the outside of the internal space.

4. The cytodiagnosis method according to claim 1,
wherein in the first injection step and the second injection step,
a space which is disposed on the front surface side of the filter is formed,
the space is surrounded by the pressing member, an inner wall surface of the chamber and the filter, and
the space in which the cell suspension and the staining solution are injected is formed in the front surface side of the filter.

5. The cytodiagnosis method according to claim 1,
wherein in the first discharge step and the second discharge step,
a space which is disposed on the back surface side of the filter is formed,
the space is surrounded by the pressing member, an inner wall surface of the chamber and the filter, and
the space in which the cell suspension and the staining solution are discharged is formed in the back surface side of the filter.

6. The cytodiagnosis method according to claim 4,
wherein in the observation step, a space between the filter and the pressing member is filled with a liquid.

7. The cytodiagnosis method according to claim 1,
wherein in the observation step, the cells on the filter are observed by the observation unit through a cover glass provided between the observation unit and the filter.

8. The cytodiagnosis method according to claim 1,
wherein in the observation step, light from the light source is transmitted through the pressing member.

9. A cytodiagnosis device comprising:
a filter having a pore size that is capable of capturing cells contained in a cell suspension and fine undulations;
a chamber having an internal space and having the filter disposed in the internal space;
an injection unit that injects the cell suspension and a staining solution for staining the cells into the internal space of the chamber from a front surface side of the filter;
a discharge unit that is configured to discharge the cell suspension and the staining solution from a back surface side of the filter to an outside of the chamber;
a pressing member that is provided in the internal space so as to be movable relative to the filter;
a control unit that is configured to control a movement of the pressing member;
a light source that is configured to irradiate the cells with light; and
an observation unit that is provided on a side opposite to the light source with respect to the filter,
wherein the control unit is configured to bring the pressing member into contact with the filter by moving the pressing member to the filter in a direction in which the pressing member and the filter approach each other, and to press the filter by the pressing member, and
the undulations of the filter are flattened.

10. The cytodiagnosis device according to claim 9,
wherein the light source is provided so as to be movable relative to the observation unit, and
the light source is configured to push any one of the pressing member and the filter in a direction approaching the other.

11. The cytodiagnosis device according to claim 9,
wherein the pressing member is provided in the chamber and is disposed on the front surface side or the back surface side of the filter, and
a space which is surrounded by the pressing member, an inner wall surface of the chamber and the filter is formed, the cell suspension and the staining solution injected into the space.

12. The cytodiagnosis device according to claim 9,
wherein the injection unit and the discharge unit are attachable to and detachable from the chamber.

13. The cytodiagnosis device according to claim 9,
wherein the pressing member is made of a light-transmitting material through which light from the light source is transmitted.

14. The cytodiagnosis device according to claim 9,
wherein the pressing member is a diffusion plate that is configured to diffuse light from the light source.

15. The cytodiagnosis device according to claim 9,
wherein a cover glass is provided between the observation unit and the filter.
